(19) United States
(12) Patent Application Publication
(10) Pub. No.: US 2025/0076428 A1
(43) Pub. Date: Mar. 6, 2025

(54) Support Element and Magnet Support Structure

(71) Applicant: Siemens Healthcare Limited, Camberley (GB)

(72) Inventor: Michael Simpkins, Finstock (GB)

(73) Assignee: Siemens Healthcare Limited, Camberley (GB)

(21) Appl. No.: 18/817,714

(22) Filed: Aug. 28, 2024

(30) Foreign Application Priority Data
Aug. 31, 2023 (EP) 23275131.3

(51) Int. Cl. G01R 33/38; A61B 5/055

(52) U.S. Cl. CPC G01R 33/3802; A61B 5/055

(57) ABSTRACT

The disclosure describes a support element for supporting a cold mass of a magnetic resonance device, comprising at least one attachment point, a plurality of displaceable sections and a plurality of fixed sections. Each displaceable section of the plurality of displaceable sections is configured to be reversibly displaced relative to fixed sections of the plurality of fixed sections when a predefined force is applied to the at least one attachment point and the fixed sections of the plurality fixed sections are configured to remain in a predefined relative spatial arrangement to each other when the predefined force is applied to the at least one attachment point. The predefined force is oriented in at least one spatial direction. The disclosure further describes a magnet support structure, comprising an outer vacuum chamber, a cold mass, and a support element, as well as a computer-implemented method for designing a support element.

### CROSS-REFERENCE TO RELATED APPLICATIONS

The present application claims priority to and the benefit of European patent application no. EP 23275131.3, filed on Aug. 31, 2023, the contents of which are incorporated herein by reference in their entirety.

### TECHNICAL FIELD

The disclosure relates to support elements and an accompanying support structure and, more particularly, to support elements and structures for supporting a cold mass of a magnetic resonance imaging device.

### BACKGROUND

Magnets for magnetic resonance devices typically operate at very low temperature levels, for example temperature levels in the range of 3 K to 5 K. In order to maintain the magnet at these low temperature levels as efficiently as possible, the magnets are suspended from a vacuum chamber. The vacuum chamber may also serve as a support structure preventing the magnet from moving when electromagnetic forces are acting on the magnet during a magnetic resonance measurement, but also during transport of the magnetic resonance device.

Recent developments have led to lightweight, serially bonded magnet coil structures. At the same time, increasing material costs and decreasing availability of cryogens used for cooling the magnets have favored the development of so called “dry” magnetic resonance devices. In a “dry” magnetic resonance device, the magnet is cooled via thermal conduction rather than being immersed in a liquid cryogen contained within a dedicated cryogen vessel. The magnets are typically suspended from the vacuum chamber using suspension elements. However, a tension that needs to be applied to these suspension elements may cause a distortion of lightweight magnets, which is detrimental to a homogeneity of magnetic fields generated by the magnets.

This problem is worsened as a magnetic field strength of the magnet, and thus a mass of the magnet, increases. For example, a low field 0.5 T magnet usually requires less tension than a 1.5 T magnet. High field magnets, such as 3 T magnets or 7 T magnets, are significantly heavier and will require an even higher tension. With increasing tension loads, a stiffness or rigidity of the support structure needs to increase as well, thus increasing the mass and overall complexity.

### SUMMARY

It is an object of the present disclosure to reduce weight, but also complexity, of magnet support structures for magnetic resonance devices.

This objective is achieved by a support element, a magnet support structure, a magnetic resonance device, and a computer-implemented method for designing a support element according to the disclosure. Further advantageous embodiments are specified in the dependent claims.

A support element is configured to support a cold mass of a magnetic resonance device. The support element comprises at least one attachment point, a plurality of displaceable sections, and a plurality of fixed sections. In an embodiment, the cold mass is mechanically connected or attached to the plurality of fixed sections.

A cold mass of the main magnet can be any structure maintained a temperature level close to a superconducting temperature of a main magnet of the magnetic resonance device. For example, the cold mass may comprise or represent the main magnet and/or a cryogen vessel of the magnetic resonance device. The cold mass may represent an inert mass.

The support element may be configured to reduce or avoid a deforming or warping of an inert mass mechanically connected to the plurality of fixed sections when a predefined force is applied to the at least one attachment point of the support element.

A main magnet of a magnetic resonance device may comprise one or more superconducting magnets. In an embodiment, the main magnet comprises one or more solenoidal or cylindrical superconducting coils. The one or more solenoidal or cylindrical superconducting coils may be rotationally symmetric or comprise rotationally symmetric bodies. According to an embodiment, each solenoidal or cylindrical superconducting coil comprises an axis of rotational symmetry arranged on an axis of rotational symmetry of the main magnet. The one or more solenoidal coils of the main magnet may be serially bonded. For example, the one or more solenoidal coils of the main magnet may be mechanically connected via material bonds and/or spacers. The main magnet may comprise a dedicated support structure configured to carry the main magnet and/or provide support to the main magnet. The term “main magnet” as used herein may comprise one or more solenoidal superconducting coils as well as a dedicated support structure.

The at least one attachment point may represent a structural feature protruding from a surface, e.g. a lateral surface, of the support element. In an embodiment, the at least one attachment point provides a mounting element, e. g. an anchor, an eyelet, a ring, a plate, a rod, a protrusion, or the like, configured for mechanically connecting the support element to a support structure, e.g. an outer vacuum chamber. The at least one attachment point may be configured to bear loads or forces associated with a transport and/or an operation of a magnetic resonance device. In an embodiment, the at least one attachment point may be configured to carry a weight of the support element and the cold mass and/or to transfer forces associated with the transport and/or operation of the magnetic resonance device from the support element to the support structure. The support element may be configured to absorb a share of the predefined force associated with the transport and/or operation of the magnetic resonance device.

According to an embodiment, the support element comprises a plurality of attachment points. The plurality of attachment points may be distributed over a surface, e.g. a lateral surface, of the support element.

In an embodiment, the at least one attachment point, the plurality of displaceable sections, and the plurality of fixed sections are mechanically connected. The support element may comprise the shape of a ring, a hollow cylinder, a hollow prism, a tube, or a disc. In an embodiment, the plurality of displaceable sections and the plurality of fixed sections are arranged along a tubular shell or wall of the support element or form parts of the tubular shell or wall of the support element. In an embodiment, displaceable sections of the plurality of displaceable sections and fixed sections of the plurality of fixed sections may be arranged along the tubular shell of the support element in an alternating fashion.

In an embodiment, a displaceable section and/or a fixed section represent a section, a part, or a segment of a ring, a hollow cylinder, a hollow prism, a tube, or a disc.

In an embodiment, the plurality of displaceable sections and the plurality of fixed sections form a connected and/or continuous structure. The plurality of displaceable sections may be directly mechanically connected to the plurality of fixed sections. For example, a displaceable section of the plurality of displaceable sections may be screwed, bolted, clamped, welded, and/or glued to a fixed section of the plurality of fixed sections. It is also conceivable that the plurality of displaceable sections and the plurality of fixed sections form a monolithic structure.

A support element shaped like a tube or prism may circumferentially encompass the cold mass of a magnetic resonance device at least partially along an axial direction defined by the cold mass. Thus, the support element may favorably support the cold mass at dedicated points distributed over a circumference of the cold mass, which may ensure an even distribution of forces associated with a transport and/or operation of the magnetic resonance device. A ring-shaped or tube-shaped support element may benefit from an increased stiffness and/or rigidity in comparison to other geometric shapes.

According to the disclosure, each displaceable section of the plurality of displaceable sections is configured to be reversibly displaced relative to fixed sections of the plurality of fixed sections when a predefined force is applied to the at least one attachment point, and the fixed sections of the plurality of fixed sections are configured to remain in a predefined spatial arrangement to each other when the predefined force is applied to the at least one attachment point.

The fixed sections may be configured to maintain a relative spatial position to each other when the predefined force is applied to the at least one attachment point.

According to an embodiment, the fixed sections are configured to maintain a relative spatial position with respect to an inert mass mechanically connected to the plurality of fixed sections when the predefined force is applied to the at least one attachment point. In an embodiment, the support element is configured to prevent the fixed elements from shifting against each other when the predefined force is applied to the at least one attachment point. Thus, a deformation of an inert mass mechanically connected the plurality of fixed sections may be reduced or avoided. The inert mass may represent a cold mass of a magnetic resonance device.

The predefined force may be oriented in at least one spatial direction. However, the predefined force may also comprise a plurality of force components oriented in different spatial directions. The predefined force may be a force associated with an acceleration and/or a deceleration of a magnetic resonance device during a transport. In an embodiment, the predefined force may represent a suspension load acting on the at least one attachment point. However, the predefined force may also be associated with a shock or impact on a housing or an outer shell of a magnetic resonance device comprising the support element. In one example, the predefined force may correspond to a gravitational force or a multiple of the gravitational force. However, the predefined force may also represent an acceleration force, a retarding force, and/or a centrifugal force acting on the at least one attachment point.

A magnitude of the predefined force may be limited to a maximum force expected to act on the attachment point during a transport of the magnetic resonance device comprising the support element. In an embodiment, the predefined force is smaller than a force required to exceed a fatigue limit or a breaking point of a material of the support element. The material of the support element may be configured to bear the predefined force over an elongated period of time, such as days, weeks, months, or years.

The plurality of displaceable sections may be configured to move, bend, and/or deform when the predefined force is applied to the at least one attachment point. According to an embodiment, the displaceable sections may be displaced by up to any suitable displacement range such as for example up to 1 mm, up to 2 mm, up to 3 mm, up to 4 mm, etc., when the predefined force is applied to the at least one attachment point. It is also conceivable that the displaceable sections are displaced by any suitable displacement range such as for example at least 1 mm, at least 2 mm, at least 3 mm, etc., when the predefined force is applied to the at least one attachment point.

The fixed sections may be configured to be displaced or moved relative to each other by a minimal distance when the predefined force is applied to the at least one attachment point. In an embodiment, a displacement of fixed sections is negligible in comparison to a displacement of the displaceable sections when the predefined force is applied to the at least one attachment point. For example, the fixed sections may be considered to remain in a predefined relative spatial arrangement to each other if a spacing or distance of two fixed sections of the plurality of fixed sections is changed by less than any suitable distance such as for example 0.5 mm, less than 0.2 mm, less than 0.1 mm, etc., when the predefined force is applied to the at least one attachment point.

The fixed sections may also be considered to remain in a predefined spatial arrangement to each other if each fixed section of the plurality of fixed sections is displaced by a minimal distance when the predefined force is applied to the at least one attachment point. A minimal distance may be significantly smaller than a displacement of a displaceable section when the predefined force is applied to the at least one attachment point. For example, the minimal distance may be any suitable distance such as for example less than 0.5 mm, less than 0.2 mm, less than 0.1 mm, etc. However, the minimal distance may also be less than any suitable proportion such as for example 15%, less than 10%, less than 5%, etc., of a displacement of a displaceable section when the predefined force is applied to the at least one attachment point.

The displaceable sections may also be considered as deformable sections. In an embodiment, a shape and/or spatial configuration of the displaceable sections may differ from a shape and/or spatial configuration of the fixed sections in such a way that the support element is deformed at sections corresponding to the displaceable sections and a deformation of fixed sections is avoided when the predefined force is applied to the at least one section. For example, a material thickness and/or a 3-dimensional shape of the displaceable sections may differ from a material thickness and/or a 3-dimensional shape of the fixed sections in such a way, that the support element primarily deforms at the displaceable sections when the predefined force is applied to the at least one section. It is also conceivable that a material composition of the displaceable sections differs from a material composition of the fixed sections. For example, the displaceable sections may comprise or consist of a material having a lower Young's modulus than a material of the fixed sections.

In an embodiment, the support element is designed in such a way to allow for a deformation or deflection of the plurality of displaceable sections remotely from the plurality of fixed sections. Thus, an inert mass mechanically connected to the plurality of fixed sections may substantially remain unaffected by the predefined force applied to the at least one attachment point.

In an embodiment, the support element is designed via a computer-implement method. The computer-implemented method for designing the support element comprises the steps:
performing a load simulation of the support element to determine a deformation pattern of the support element when a predefined force is applied to at least one attachment point, wherein an inert mass is mechanically connected to the support element at a plurality of sections arranged at predefined spatial locations, and
determining a parameter of the support element based on the deformation pattern to provide a support element comprising a plurality of displaceable sections and a plurality of fixed sections arranged at the predefined spatial locations.

The load simulation may comprise a numerical simulation and/or finite elements simulation. The deformation pattern may comprise an information on a displacement of one or more discrete points or volume elements of the support element.

The deformation pattern may depend on the applied predefined force, but also on properties of the support element and/or the inert mass, such as a weight, a shape, and/or a mechanism used to attach the inert mass to the support element. The deformation pattern may be used to modify and/or determine a parameter or a set of parameters of the support element, such as a shape, a material composition, and/or a spatial configuration. In an embodiment, the parameter or set of parameters may be modified in such a way to provide a support element comprising a plurality of fixed sections arranged at predefined or desired spatial locations. The predefined or desired spatial locations of the plurality of fixed sections may depend on a shape and/or a mechanical configuration of the inert mass. In an embodiment, the inert mass corresponds to a cold mass of a magnetic resonance device.

The computer-implemented method may be used to optimize the support element with respect to a weight and/or a size of the support element, but also with respect to spatial locations of the plurality of fixed sections. For example, the computer-implemented method may comprise a step of iteratively modifying a parameter or a set of parameters of the support element in dependence of one or more determined deformation patterns. In an embodiment, the optimization of the support element is based on a cost-function, a least-squares method, a Newton's method, a Gauss-Newton algorithm, a Lagrange multiplier, or the like.

According to an alternative embodiment, a load simulation may be used to identify a plurality of sections of the support element which exhibit a minimum displacement when a predefined force is applied to at least one attachment point and when the inert mass is mechanically connected to at least one section of the support element.

According to an embodiment of the support element, the plurality of fixed sections is configured to prevent or reduce a deformation of an inert mass connected to the plurality of fixed sections when the predefined force is applied to the at least one attachment point. A support element may favorably allow for suspension loads acting on the at least one attachment point to be deflected to regions of the support element (i. e. the plurality of displaceable sections) which are configured to be elastically deformed. Thus, a deformation of an inert mass, e.g. a main magnet and/or a cryogen vessel, carried by the support element may favorably be reduced or avoided.

Conventional solutions for supporting the cold mass of a magnetic resonance device tend to be rigid, heavy, and somewhat complex, which increases manufacturing costs, but may also complicate transport. In providing a support element configured to prevent a displacement or deflection of fixed sections mechanically connected to the cold mass, material costs as well as manufacturing efforts associated with the magnet support structure may favorably be reduced.

Precise manufacturing techniques may need to be applied only to a few key locations of the support element, e.g. where an inert mass is to be attached to the support element or where the support element is to be attached to a support structure. Thus, large portions of the support element may favorably be processed using relatively simple and low-cost manufacturing processes such as water jet cutting or casting.

According to an embodiment of the support element, the at least one attachment point is arranged at a displaceable section of the plurality of displaceable sections and mechanically connected to the displaceable section.

As described above, the at least one attachment point may be configured to mechanically connect to a support structure, which may apply or transfer suspension loads and/or other forces to the at least one attachment point. In an embodiment, the at least one attachment point is configured to introduce the suspension loads and/or other forces into the support element.

The at least one attachment point may protrude from a surface of a displaceable element of the plurality of displaceable elements. In an embodiment, the at least one attachment point protrudes outward from a surface of the support element, e. g. along a radial direction of the support element or a direction leading away from the support element.

In providing a support element comprising at least one attachment point attached to a displaceable section, the at least one attachment point is allowed to move or bend together with the displaceable section when a suspension load and/or another force is applied to it. Thus, requirements regarding a material choice and/or a rigidity of the at least one attachment point, but also the support element, may favorably be reduced.

In an embodiment, the support element comprises at least one connecting element configured to mechanically connect to an inert mass. The at least one connecting element may be embodied in accordance with a connecting element described below. An inert mass may correspond to a cold mass of a magnetic resonance device. In an embodiment, the at least one connecting element is configured to carry the inert mass or suspend the inert mass from the support element.

According to the disclosure, the at least one connecting element is arranged at a fixed section of the plurality of fixed sections and mechanically connected to the fixed section. The at least one connecting element may be mechanically connected to the support element according to an embodiment described below. However, the support element and the at least one connecting element may also form a monolithic structure.

According to an embodiment, the support element comprises an inert mass, e.g. a cold mass of a magnetic resonance device, mechanically connected to the at least one connecting element.

The at least one connecting element may favorably allow for mechanically connecting an inert mass to the support element. As the at least one connecting element is arranged at a fixed section, a deformation or torsion of the inert mass may favorably be reduced or avoided, when the predefined force is applied to the at least one attachment point.

According to an embodiment of the support element, the plurality of displaceable sections comprises a first displaceable section and a second displaceable section. The first displaceable section and the second displaceable section are separated by a fixed section of the plurality of fixed sections.

For example, the fixed section may be arranged between the first displaceable section and the second displaceable section. It is also conceivable that the first displaceable section and/or the second displaceable section are each arranged between two fixed sections of the plurality of fixed sections. According to an embodiment, the plurality of fixed sections and the plurality of displaceable sections may be arranged alternatingly along a segment of the support element. In an embodiment, the number of displaceable sections corresponds to the number of fixed sections.

In providing an alternating arrangement of displaceable sections and fixed sections, mechanically connecting the support element to rotationally symmetric objects of the cold mass, such as the main magnet or the cryogen vessel, may favorably be facilitated. For example, the main magnet may be mechanically connected to different points along an inner circumference of a tubular or ring-shaped support element, thus decreasing a tendency of rolling or twisting of the main magnet in relation to the support element.

According to a further embodiment, the support element comprises at least three displaceable sections and at least three fixed sections.

In an embodiment, the at least three displaceable sections and the at least three fixed sections are alternatingly distributed along a segment of the support element, e.g. a wall or shell of a tubular or ring-shaped support element. The at least three displaceable sections and the at least three fixed sections may be arranged in regular or irregular intervals along the shell of the support element.

A dimension of a displaceable section of the plurality of displaceable sections may differ from a dimension of a fixed section of the plurality of fixed sections. In an embodiment, a dimension of a displaceable section of the plurality of displaceable sections may exceed a dimension of a fixed section of the plurality of fixed sections.

In increasing a number of fixed sections mechanically connected to the cold mass, a tendency of rolling or twisting of the cold mass due to electromagnetic forces occurring during an operation of a magnetic resonance device may favorably be reduced.

According to a further embodiment, the support element comprises at least three attachment points, e.g. four attachment points.

It is conceivable that each displaceable section of the plurality of displaceable sections comprises exactly one attachment point. However, it is also conceivable that multiple attachment points are arranged at one displaceable section of the plurality of displaceable sections.

According to an embodiment, a first attachment point and a second attachment point are arranged at a first displaceable section of the plurality of displaceable sections, whereas a third attachment point and a fourth attachment point are arranged at a second displaceable section of the plurality of displaceable sections. The plurality of displaceable sections of the support element may comprise a third displaceable section which is spaced or separated from the first attachment point and the second attachment point, but also from any other attachment point of the support element.

In providing a plurality of attachment points configured to mechanically connect to a support structure, a tendency of twisting or rolling of the support element relative to the support structure may favorably be reduced or avoided.

According to an embodiment, the support element comprises the shape of a tube, a ring, or a disc, and the at least one attachment point protrudes from a lateral surface of the support element.

A lateral surface of the support element may correspond to an outer surface of the support element. For example, the support element may comprise the shape of a hollow cylinder and the lateral surface may correspond to an outer surface of the hollow cylinder.

The at least one attachment point may be configured according to an embodiment described above. The at least one attachment point may be mechanically connected to the lateral surface of the support element via a form-locking, a force-locking, and/or a material connection. For example, the attachment point may be screwed, bolted, clamped, swaged, welded, and/or glued to the support element. In an embodiment, the at least one attachment point and the support element, e.g. a displaceable section of the support element, form a monolithic structure.

In providing a support element comprising at least one attachment point according to an embodiment described above, a mechanical connection between the support element and a support structure carrying the support element may favorably be facilitated.

In an embodiment, the support element comprises at least two attachment points oriented at opposing sides of the support element.

A side of the support element may correspond to a segment of a ring, a hollow cylinder, or a tube of the support element. An opposing side may represent a side of the support element arranged at any suitable rotational angle of for example, 160° to 200°, 180°, etc., with respect to a reference side of the support element. For example, a second side arranged at 180° with respect to a first side along a circumference of the support element may be regarded as opposing the first side.

In a further example, the support element comprises a first attachment point and a second attachment point. The first attachment point may be arranged on a first side of the support element and the second attachment point may be arranged on a second side of the support element. The first attachment point may be arranged symmetrically to the second attachment point. In an embodiment, the first attachment point may be arranged at a first side of the support element and the second attachment point may be arranged at a second side of the support element opposing the first side. It is conceivable that the first attachment point and the second attachment are arranged along a perpendicular to the axis of the support element.

In an embodiment, the support element comprises two attachment points arranged at a first side of the support element and two further attachment points arranged at a second side of the support element opposing the first side.

In arranging a plurality of attachment points on two opposing sides of the support element, a distribution of the plurality of fixed sections over a circumference of the support element may be facilitated and/or homogenized. In an embodiment, a more uniform distribution of the plurality of fixed points over the circumference of the support element may be achieved. Furthermore, arranging a plurality of attachment points on two opposing sides of the support element may allow for the suspension elements to be longer, which may favorably reduce or minimize a transport of heat energy to the main magnet along the suspension elements. As a further advantage, the support element may allow for an angle between the suspension elements and a gravitational force vector to be modified in such a way to reduce or minimize distortion of the suspension elements and the outer vacuum chamber, but also restrain the main magnet from rotational movement.

According to an embodiment, the support element comprises the shape of a tube or a ring and the plurality of displaceable sections is configured to be displaced along a radial direction of the support element when the predefined force is applied to the at least one attachment point. A vector of the predefined force is oriented in parallel to a vector of the gravitational force. The predefined force may correspond to a gravitational force acting on the at least one attachment point. A gravitational force acting on the at least one attachment point may represent a standard load scenario of the magnetic resonance device.

In an embodiment, the at least one displaceable section is configured to be displaced away from a geometric center of the support element when the predefined force is applied to the at least one attachment point. However, it is also conceivable that at least one displaceable section is configured to be displaced towards a geometric center of the support element when the predefined force is applied to the at least one attachment point force.

The plurality of displaceable sections may be bend and/or comprise a variable material thickness to allow for a deformation or displacement along the radial direction of the support element.

In allowing the plurality of displaceable sections to be displaced along a radial direction of the support element, a deformation and/or or motion of the plurality of displaceable sections may favorably be compensated by a gap provided between the plurality of displaceable elements and a cold mass mechanically connected to the support element.

In allowing the plurality of displaceable sections to be displaced along the radial direction of the support element instead of an axial direction of the support element, a twisting or swaying motion of the support element may favorably be avoided. Furthermore, a displacement of the plurality of displaceable sections along a radial direction of the support element may favorably allow for arranging multiple support elements along an axial direction of a main magnet or a cryogen vessel without causing a mechanical interference between adjacent support elements.

According to an embodiment, the plurality of displaceable sections is configured to be displaced along a radial and/or an axial direction of the support element when the predefined force is applied to the at least one attachment point. In providing a plurality of displaceable sections configured to be displaced along a radial and an axial direction of the support element, the force applied to the at least one attachment point may be absorbed or deflected with lesser overall displacement of the plurality of displaceable sections in a specific direction.

The magnet support structure comprises an outer vacuum chamber, a cold mass, and a support element according to an embodiment described above.

The outer vacuum chamber may form a vessel which is substantially impermeable to fluids, such as a liquid or a gaseous cryogen. The vacuum chamber may be configured to maintain a vacuum in an inner volume encompassed by the vacuum chamber. In an embodiment, the outer vacuum chamber encompasses other components of the magnet support structure, such as a cryogen vessel, a main magnet, the support element, a thermal shield, and the like. According to an embodiment, the outer vacuum chamber comprises an outer shell, an inner shell and annular end walls connecting the outer shell and the inner shell. For example, the outer vacuum chamber may form a double-walled hollow cylinder enclosing the main magnet and the support element between the outer shell, the inner shell and the annular end walls. The outer vacuum chamber may comprise an axis of rotational symmetry oriented in parallel to or corresponding to an axis of rotational symmetry defined by the main magnet, e.g. a solenoid coil of the main magnet. The inner shell of the outer vacuum chamber may correspond to a patient bore of a magnetic resonance device.

According to the disclosure, the at least one attachment point is mechanically connected to the outer vacuum chamber.

In an embodiment, the outer vacuum chamber is configured to provide mechanical support to the support element. For example, the support element may be suspended of or carried by the outer vacuum chamber. In an embodiment, the at least one attachment point is mechanically connected to the outer vacuum chamber via at least one suspension element, e.g. a plurality of suspension rods. The support structure may comprise a plurality of attachment points, each attachment point being mechanically connected to the outer vacuum chamber via a suspension element. The suspension element may for example consist of a material with high tensile strength. Examples for suitable materials are metals, e.g. stainless steel, or composite materials, such as fiber reinforced plastics, and the like. The suspension element may comprise the shape of a bar, a rod, a pole, but also a band or a wire. A first end and a second end of the suspension element may each comprise an anchor or ferrule. The anchor or ferrule may be attached to the outer vacuum chamber and/or the support element via a suitable mechanical connection, such as a form-locking connection, a force-locking connection, and/or a material bond. For example, the anchor or ferrule may be screwed, bolted, clamped, wedged, and/or glued to the outer vacuum chamber and/or the support element. However, any suitable mechanical connection may be used.

In providing a support element suspended of the outer vacuum chamber via a plurality of suspension elements, a combined weight force of the support element and the cold mass may favorably be compensated so that balanced suspension forces are achieved at dedicated attachment points.

A suspension element according to an embodiment described above may favorably comprise a small or minimized cross-sectional-area. Thus, a transfer of heat energy along the suspension element, but also a weight of the suspension elements, may favorably be reduced.

The plurality of fixed sections is mechanically connected to the cold mass.

The plurality of fixed sections may be connected to the cold via any suitable mechanical connection, e.g. a form-locking, a force-locking, and/or a materially connection. For example, the plurality of fixed may be screwed, bolted, clamped, and/or glued to the cold mass. However, it is also conceivable that at least one connecting element is provided between the plurality of fixed sections and the cold mass. The connecting element may be mechanically connected to a fixed section of the plurality of fixed sections and the cold mass via any suitable mechanical connection.

According to the disclosure, the cold mass is spaced from the plurality of displaceable sections.

In an embodiment, the connecting element is configured to space the plurality of fixed sections from the cold mass. An inner diameter of the support structure may exceed a diameter of the cold mass. For example, an inner diameter of a tubular support structure may exceed an outer diameter of the main magnet and/or an outer diameter of the cryogen vessel. Thus, one or more gaps may be provided between sections of the support element and the cold mass, which may allow for the plurality of displaceable sections to move or bend without contacting the support element.

The magnet support structure shares the advantages of an support element according to an embodiment described above.

According to an embodiment of the magnet support structure, the plurality of fixed sections is mechanically connected to the cold mass via a plurality of connecting elements. The plurality of connecting elements is configured to space the support element from the cold mass.

A connecting element may provide a mechanical connection between a fixed section and the cold mass. The connecting element may be a simple spacer, such as a pin, a bolt, a block, a plate, a ring, a segment of a ring, a hollow cylinder, a sector of a hollow cylinder, or the like. However, the connecting element may also represent a suspension rod. It is conceivable that the connecting element is mechanically connected to the support element and/or the cold mass via a form-locking, a force-locking and/or a materially connection.

In an embodiment, the plurality of connecting elements comprises at least two connecting elements spaced apart along a circumference of the cold mass in such a way, that a gap is provided between the at least two connecting elements, but also between the support element and the cold mass. According to an embodiment, the plurality of connecting elements comprises at least three connecting elements which are spaced apart along a circumference of the cold mass in such a way, that gaps are provided between the at least three connecting elements along the circumference of the cold mass. In an embodiment, a location of a gap between two connecting elements of the plurality of connecting elements coincides with a location of a displaceable section along a circumference of the support element. For example, gaps may be provided between the cold mass and each displaceable section of the plurality of displaceable sections.

In providing gaps between the support element and the cold mass, a contact between the plurality of displaceable elements and the cold mass may favorably be avoided when the at least one attachment point is loaded with a predefined force.

The magnetic resonance device is configured for acquiring magnetic resonance data of an object positioned within an imaging region of the magnetic resonance device.

In an embodiment, the magnetic resonance device is configured to acquire magnetic resonance image data, e.g. diagnostic magnetic resonance image data, from the object positioned within the imaging region. The object may be a patient, e.g. a human or an animal.

The magnetic resonance device may represent a closed bore scanner. A closed bore scanner may comprise a substantially cylindrical bore circumferentially enclosing the imaging region. The main magnet of the closed bore scanner may comprise one or more solenoid coils circumferentially encompassing the imaging region along an axial direction or an axis of rotational symmetry of the cylindrical bore. The one or more solenoid coils may comprise a wire having negligible electrical resistance at (or below) a superconducting temperature. A direction of a main magnetic field provided via the main magnet may be oriented substantially in parallel to a direction of access of the object to the imaging region and/or the axial direction of the cylindrical bore.

The magnetic resonance device may represent a “dry” system comprising a minimum of cryogen or no cryogen at all. For example, the magnetic resonance system may comprise one or more small cryogen vessels thermally connected to the main magnet via a solid thermal conductor. The small cryogen vessels may contain a volume of less than any suitable volume such as for example less than 10 liters, less than 5 liters, or less than 1 liter, etc., of cryogen. In an embodiment of the magnetic resonance device, a cryogen vessel is omitted. Thus, the main magnet may be cooled entirely via thermal conduction.

It is conceivable that the components of the magnetic resonance device, such as the main magnet, the support element, the magnet support structure, the thermal shield, one or more suspension elements, the cryogen vessel, or the like, are thermally connected to the at least one cryocooler, e.g. via a solid thermal conductor, a convection loop, and/or a heat pipe.

In a “dry” system, the term cold mass may refer to the main magnet. For example, the main magnet may be mechanically connected to the plurality of fixed sections of the support element according to an embodiment described above. For example, the main magnet may be cooled exclusively via thermal conduction. However, a “dry” magnetic resonance device may still comprise one or more cryogen vessels comprising a cryogen thermally connected to the main magnet. According to an embodiment, the magnetic resonance device comprises a cryogen vessel containing a cryogen, wherein the cryogen vessel is thermally connected to the main magnet via a solid thermal conductor.

In an alternative embodiment, the magnetic resonance device is configured as a “wet” system. A “wet” system may comprise a cryogen vessel containing a fluid or a cryogen with a low boiling point like Argon, Nitrogen, Neon, Helium, or the like. In a “wet” system, the main magnet may be disposed within the cryogen vessel and at least partially immersed in a liquid portion of the cryogen. The term cold mass may refer to the cryogen vessel. For example, the cryogen vessel may be mechanically connected to the plurality of fixed sections of the support element according to an embodiment described above. Alternatively, the support element and the main magnet may be mechanically supported within the cryogen vessel. In this case, the term cold mass may refer to the main magnet, which is mechanically connected to the plurality of fixed sections of the support element according to an embodiment described above.

The magnetic resonance device may comprise at least one cryocooler. The at least one cryocooler may be configured to cool components of the magnetic resonance device. For example, the at least one cryocooler may be configured to cool the main magnet, a thermal shield, the cryogen vessel, parts of the magnet support structure, and the like.

The at least one cryocooler may be configured to provide a temperature close to or lower than a superconducting temperature of a superconducting material of the main magnet. For example, the superconducting temperature of the main magnet may be in any suitable temperature range such as 3 K to 100 K, 3 K to 5 K, 30 K to 60 K, 60 K to 90 K, etc. The at least one cryocooler may be implemented as a pulse tube refrigerator, a Gifford-McMahon refrigerator, a Stirling cryocooler, a Joule-Thomson cryocooler, or the like. In an embodiment, the at least one cryocooler comprises at least two cooling stages having different temperature levels. Thus, the cold mass and the magnet support structure of the magnetic resonance device may be maintained at different temperature levels.

In an embodiment, the at least one cryocooler is thermally and mechanically connected to the main magnet, the thermal shield, and/or a component of the magnet support structure, e.g. the support element. The at least one cryocooler may be configured to maintain the main magnet, the thermal shield, and the component of the magnet support structure at different temperature levels.

It is conceivable that components of the magnetic resonance device, such as the main magnet, the cryogen vessel, the thermal shield, the magnet support structure, and the like, are thermally connected to the cryocooler. In an embodiment, the components of the magnetic resonance device are thermally connected to the cryocooler via a solid thermal conductor and/or a heat pipe.

A thermal shield may be configured to reduce a transport of heat energy to the main magnet. A transport of heat energy may be characterized by a heat transport mechanism such as thermal radiation, heat conduction, but also heat convection. The thermal shield may circumferentially encompass the main magnet. For example, the thermal shield may form a vessel enclosing the main magnet. In an embodiment, the thermal shield comprises an outer wall, an inner wall, and annular end walls connecting the outer wall and the inner wall. In an embodiment, the thermal shield may form a double-walled hollow cylinder enclosing the main magnet, but also the support element, between the outer wall, the inner wall and the annular end walls. A cylinder axis of the thermal shield may be oriented in parallel to or correspond an axis of rotational symmetry defined by the solenoidal coil of the main magnet.

In an embodiment, the thermal shield comprises or consists of a material with high electrical and/or high thermal conductivity. For example, the thermal shield may consist of copper or aluminum, e.g. a copper alloy or an aluminum alloy. The thermal shield may also comprise gold, platinum, silver, or other materials with high thermal conductivity. In one embodiment, the thermal shield is coated or galvanized with gold, platinum, or other metals with high thermal conductivity.

The thermal shield may comprise a continuous or coherent surface. In an alternative embodiment, the thermal shield may comprise or consist of a plurality of spaced shield elements or rings. The thermal shield may further comprise one or more sections of coiled wire.

In an embodiment, the thermal shield is shaped in such a way to conform to a shape of the main magnet. For example, a surface contour of the thermal shield may imitate or conform to a surface contour of the main magnet.

The magnetic resonance device comprises a magnet support structure according to an embodiment described above.

The magnetic resonance device shares the advantages of the magnet support structure and the support element according to an embodiment described above.

According to an embodiment of the magnetic resonance device, a smallest angle between a line defined by an axial extension of the at least one suspension rod and a gravitational force vector is at least 35°.

The gravitational force vector may be oriented in parallel to a surface normal of a ground surface on which the magnetic resonance device is positioned or installed.

A smallest angle may be independent from a direction of measurement and/or from a coordinate system. For example, the smallest angle may quantify a minimum angle between the line defined by an axis and/or axial extension of the at least one suspension rod and the gravitational force vector, regardless of a rotation or orientation of a coordinate system.

According to an embodiment, the smallest angle between the line defined by the axis of the at least one suspension rod and the gravitational force vector is at least 35°. In an embodiment, the smallest angle between the line defined by the axis of the at least one suspension rod and a gravitational force vector may be at least any suitable angle such as for example 40°, 45°, 50°, or 55°, etc.

In providing a magnetic resonance device comprising a magnet support structure suspended via a suspension rod oriented at a shallow angle with respect to a gravitational force vector, a rotation of the magnet support structure about a direction defined by the gravitational force vector may favorably be reduced or avoided. Furthermore, a process of mounting or attaching the support element to the outer vacuum chamber may favorably be facilitated.

### BRIEF DESCRIPTION OF THE DRAWINGS

Further advantages and details of the present disclosure may be recognized from the embodiments described below as well as the drawings. The figures show:

FIG. 1 illustrates a schematic representation of an example magnetic resonance device, in accordance with an embodiment of the present disclosure;

FIG. 2 illustrates a schematic representation of an example magnetic resonance device, in accordance with an embodiment of the present disclosure;

FIG. 3 illustrates a schematic representation of an example magnetic resonance device, in accordance with an embodiment of the present disclosure;

FIG. 4 illustrates a schematic representation of an example magnet support structure, in accordance with an embodiment of the present disclosure;

FIG. 5 illustrates a schematic representation of an example magnet support structure, in accordance with an embodiment of the present disclosure; and

FIG. 6 illustrates a schematic representation of an example support element, in accordance with an embodiment of the present disclosure.

### DETAILED DESCRIPTION OF THE DISCLOSURE

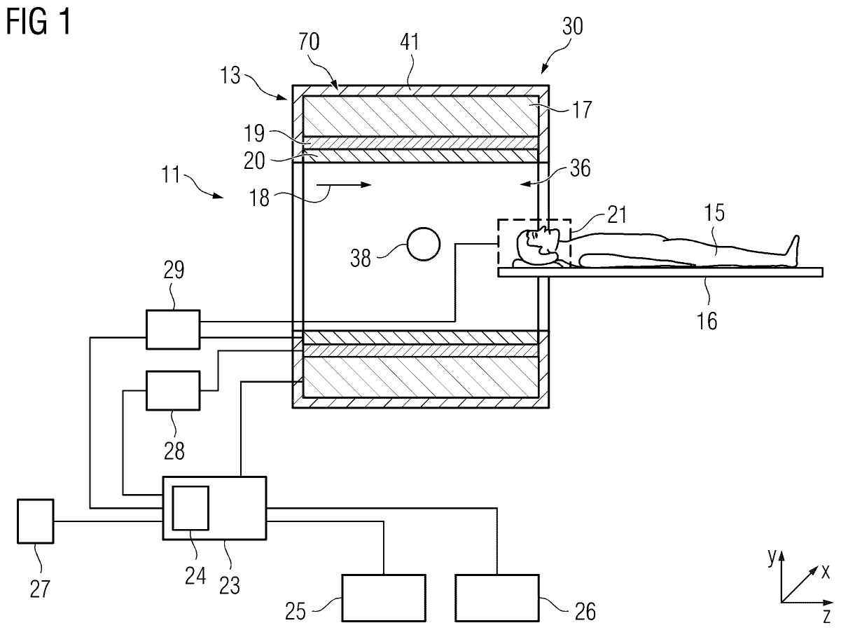

FIG. 1 shows an embodiment of a magnetic resonance device 11 according to the disclosure. In the illustrated example, the magnetic resonance device 11 comprises a static field magnet or main magnet 17 configured to provide a homogenous, static magnetic field 18 (BO field) having an imaging volume 38. The static magnetic field 18 permeates an imaging region 36 configured for receiving an imaging object, such as a patient 15. The imaging region 36 may correspond to a patient bore configured for accommodating a patient during a magnetic resonance measurement. The imaging region 36 is encompassed by the main magnet 17 in a circumferential direction.

In the depicted example, the magnetic resonance device 11 comprises a patient support 16 configured to transport the patient 15 into the imaging region 36. In an embodiment, the patient support 16 is configured to transport a diagnostically relevant body region of the patient 15 into the imaging volume 38 or isocentre of the magnetic resonance device 11. The main magnet 17 and other components of a field generation unit (not shown) of the magnetic resonance device 11 may be concealed in a housing 41.

The magnetic resonance device 11 may comprise a gradient system 19 configured to provide gradient magnetic fields used for spatial encoding of magnetic resonance signals acquired during a magnetic resonance measurement. The gradient system 19 is activated or controlled by a gradient controller 28 via an appropriate current signal. It is conceivable that the gradient system 19 comprises one or more gradient coils configured to generate gradient magnetic fields in different, e.g. orthogonally oriented, spatial directions.

The magnetic resonance device 11 may comprise an integrated radiofrequency (RF) antenna 20 (e.g. a body coil). The RF antenna 20 may be operated via a RF controller 29 that controls the RF antenna 20 to generate a high frequency magnetic field and emit RF excitation pulses into the imaging region 36. The magnetic resonance device 11 may further comprise a local coil 21. The local coil 21 may be positioned on or in proximity to the diagnostically relevant region of the patient 15. The local coil 21 may be configured to emit RF excitation pulses into the patient 15 and/or receive magnetic resonance signals from the patient 15. It is conceivable, that the local coil 21 is controlled via the RF controller 29.

In an embodiment, the magnetic resonance device 11 comprises a control unit 23 configured for controlling the magnetic resonance device 11. The control unit 23 may comprise a processing unit 24 configured to process magnetic resonance signals and reconstruct magnetic resonance images. The processing unit 24 may also be configured to process input from a user of the magnetic resonance device 11 and/or provide an output to a user. For this purpose, the processing unit 24 and/or the control unit 23 can be connected to a display unit 25 and an input unit 26 via a suitable signal connection. For a preparation of a magnetic resonance measurement, preparatory information, such as imaging parameters or patient information, can be provided to the user via the display unit 25. The input unit 26 may be configured to receive information and/or imaging parameters from the user.

Of course, the magnetic resonance device 11 may comprise further components and/or functions that magnetic resonance devices usually offer. The general operation of a magnetic resonance device 11 is known to those skilled in the art, so a more detailed description is omitted.

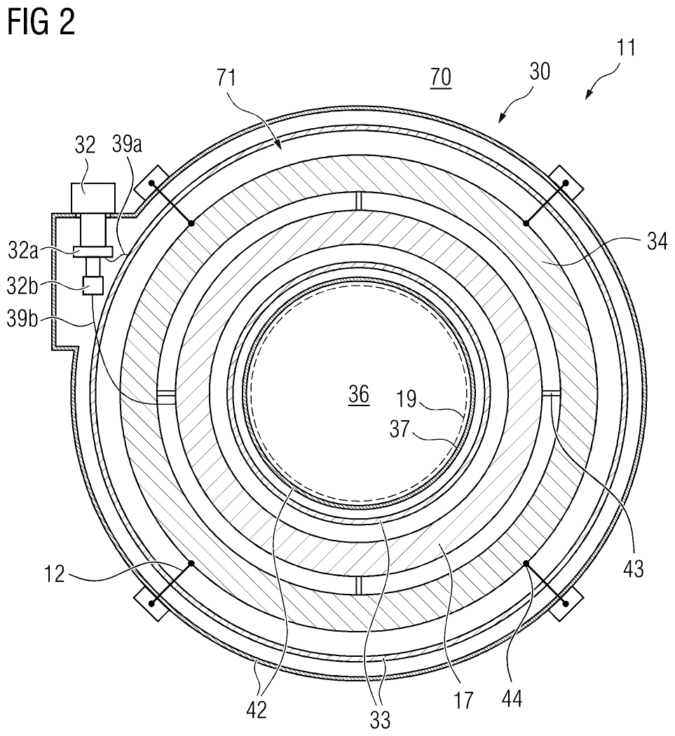

FIG. 2 shows a sectional view of the example magnetic resonance device 11 according to FIG. 1. In the depicted example, an outer vacuum chamber 42 provides an enclosure for the components of the magnet support structure 30. The outer vacuum chamber 42 separates a surrounding environment 70 from a vacuum region 71 encompassed by the outer vacuum chamber 42. In an embodiment, the outer vacuum chamber 42 forms a double-walled hollow cylinder comprising an outer shell and an inner shell. The components of the magnet support structure 30 may be housed within or enclosed by the outer shell and the inner shell of the outer vacuum chamber 42. The inner shell of the outer vacuum chamber 42 may form the patient bore 37 encompassing the imaging region 36 in a circumferential direction.

In the illustrated embodiment, the magnet support structure 30 comprises the outer vacuum chamber 42, the support element 34 (also referred to herein as a support assembly), the four suspension rods 12, and the three connecting elements 43. The support element 34 and the main magnet 17 are enclosed between an outer wall and an inner wall of a thermal shield 33. Furthermore, the main magnet 17 is arranged between the support element 34 and the inner wall of the thermal shield 33 at a transverse section through the magnetic resonance device 11, as depicted in FIG. 2.

In an embodiment, the outer shell of the vacuum chamber 42 is mechanically connected to the support element 34 via a plurality of suspension rods 12. The suspension rods 12 may extend through passages or holes in the thermal shield 33 to provide a mechanical connection between the outer shell of the outer vacuum chamber 42 and the support element 34.

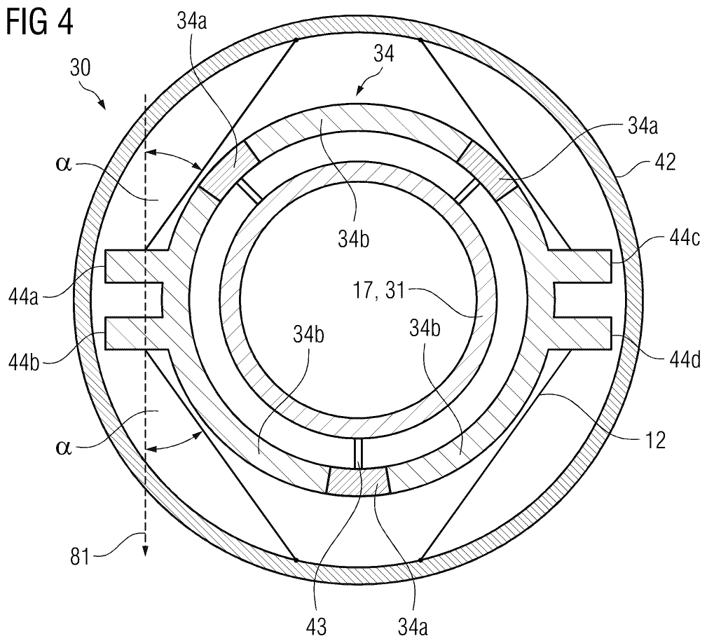

In an embodiment, the magnetic resonance device 11 comprises a cryocooler 32 mounted on the outer vacuum chamber 42. The cryocooler 32 may be configured to cool the main magnet 17, the thermal shield 33, the support element 34, but also other components of the magnet support structure 30, such as a cryogen vessel 31 (see FIG. 4).

The cryocooler 32 typically comprises a compressor supplying pressurized gas to the cryocooler 32 (not shown). According to the embodiment shown in FIG. 2, the cryocooler 32 includes a cold head comprising a plurality of cooling stages 32a and 32b. In an embodiment, a first cooling stage 32a of the cold head is thermally connected to the thermal shield 33, whereas a second cooling stage 32b of the cold head is thermally connected to the main magnet 17. According to an embodiment, the first cooling stage 32a provides a temperature level of about 50 K, for example, whereas the second cooling stage 32b provides a temperature level of about 4 K. In “dry” systems, the cooling stages 32a and 32b of the cryocooler 32 can be thermally connected to the thermal shield 33 and the main magnet 17 via solid thermal conductors 39a and 39b. It is also conceivable that the magnetic resonance device 11 comprises one or more small cryogen vessels (not shown) thermally connected to the cryocooler 32 via a solid thermal conductor, a heat pipe, and/or a convective loop. The cryogen vessel may be thermally connected to the main magnet 17 via a heat exchanger and/or a solid thermal conductor 39.

During operation of the magnetic resonance device 11, the thermal shield 33 may be maintained at an intermediate temperature, e. g. a temperature level between 40 K and 60 K, a temperature level of about 50 K, etc. The thermal shield 33 may comprise an electrically conductive material configured for shielding the main magnet 17 from thermal radiation, but also from stray fields of the gradient magnetic fields generated via the gradient system 19.

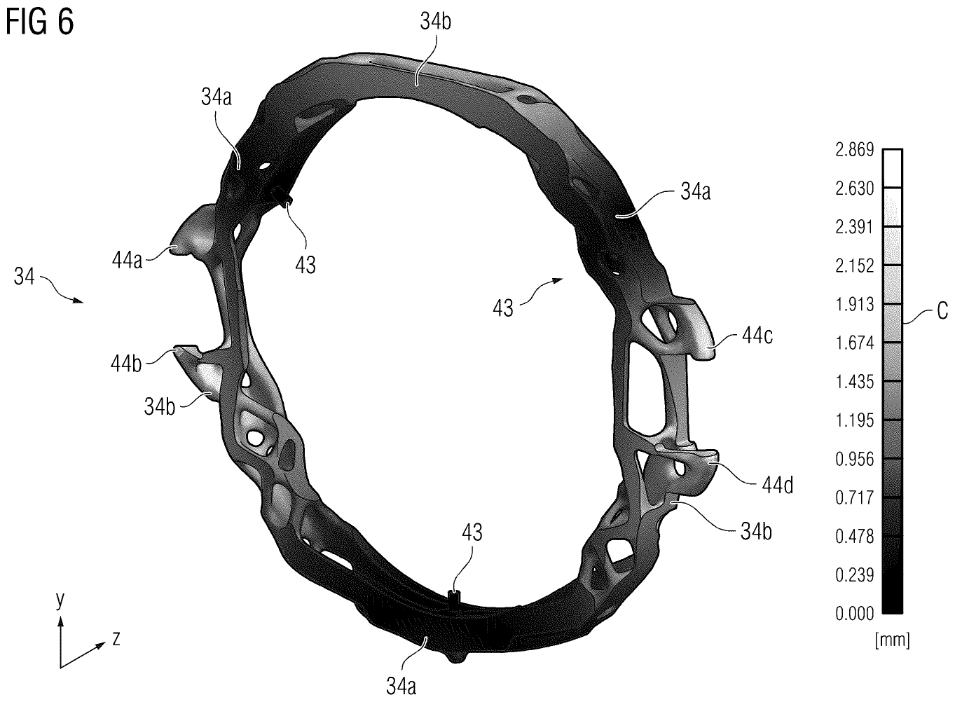

In FIG. 2, the main magnet 17 is mechanically connected to the support element 34 via four connecting elements 43 (also referred to herein as connectors). The connecting elements 43 are spaced from each other along a circumference of the main magnet 17. Thus, gaps are provided between the connecting elements 43. In the depicted example, the gaps coincide with displaceable sections of the support element 34 (see FIG. 6), which can be deformed and/or displaced without contacting the main magnet 17 and/or causing deformations near the fixed sections in proximity to the connecting elements 43. The connecting elements shown in FIG. 2 may be implemented as pins or bolts for example. In an embodiment, the connecting elements 43 are configured to carry or hold the main magnet 17 in a predefined position while spacing the main magnet 17 from the support element 34.

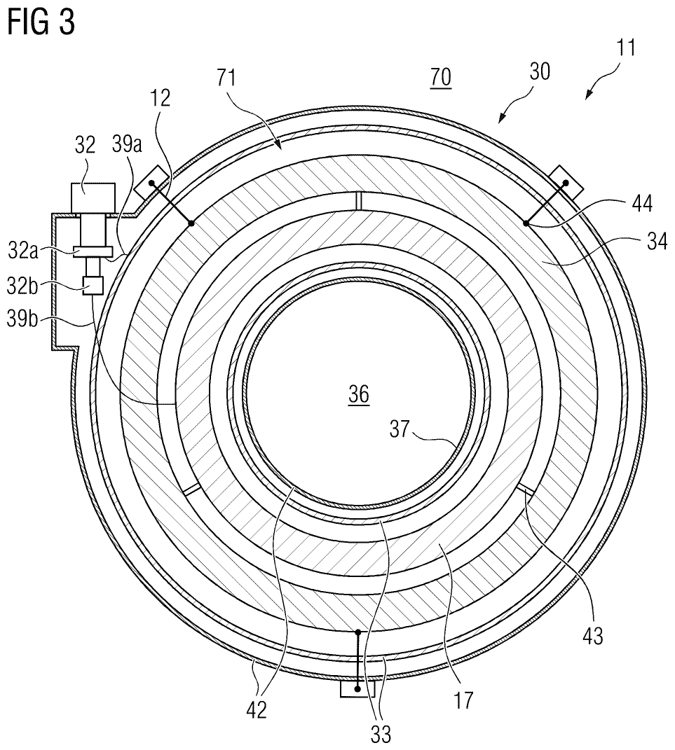

In the embodiment depicted in FIG. 3, the magnet support structure 30 comprises three suspension rods 12 mechanically connecting the outer shell of the outer vacuum chamber 42 to the support element 34. The support element 34 comprises three connecting elements 43 mechanically connected to the main magnet 17. As described in context with FIG. 2, the connecting elements 43 may for example be arranged in proximity to the fixed sections of the support element 34 to avoid a transfer of motion and/or forces from the support element 34 to the main magnet 17 when a force is applied to the attachment points 44 of the support element 34 (see FIG. 4).

In the example depicted in FIGS. 2 and 3, the number of the suspension rods 12 corresponds to the number of connecting elements 3. Likewise, the number of fixed sections may correspond to the number of displaceable sections. However, it is conceivable that the number of suspension rods 12 is dissimilar from the number of connecting elements 43. Furthermore, the number of fixed sections may differ from the number of displaceable sections.

FIG. 4 illustrates an embodiment of a magnet support structure 30 for use in a “wet” magnetic resonance device. In the depicted example, the magnet support structure 30 comprises an outer vacuum chamber 42, a support element 34 and a cryogen vessel 31 containing the main magnet 17. The support element 34 is suspended of the outer vacuum chamber 42 via suspension rods 12. The cryogen vessel 31 is mechanically connected to the support element 34 via three connecting elements 43. The main magnet 17 is mechanically supported within the cryogen vessel 31 (not shown).

In an embodiment, the magnet support structure 30 depicted in FIG. 4 is used in a “wet” magnetic resonance device. However, instead of a cryogen vessel 31, the support element 34 may be directly connected to a main magnet 17. Such a configuration of the magnet support system 30 may e.g. be used in “dry” magnetic resonance devices.

In the embodiment depicted in FIG. 4, a plurality of attachment points 44 protrude from a lateral surface of the support element 34. The attachment points 44a, 44b and the attachment points 44c, 44d are arranged at opposite sides of the support element 34. The suspension rods 12 provide a mechanical connection between the outer shell of the vacuum chamber 42 and the attachment points 44. The suspension rods 12 are oriented at a smallest angle α with respect to the gravitational force vector 81. In the depicted embodiment, the angle α is at least 35°.

The attachment points 44 may be formed as anchors. The end sections of the suspension rods 12 may comprise ferrules (not shown) configured to mechanically engage with the attachment points 44. For example, the end sections of the suspension rods 12 may comprise a male thread configured to engage with a female thread of the attachment points 44. However, any suitable mechanical connection may be used to mechanically connect the suspension rods 12 to the attachment points 44.

The support element 34 comprises three fixed sections 34a and three displaceable sections 34b. The displaceable sections 34b are configured to be bend or displaced when a force, e.g. a suspension load, is applied to at least one attachment point 44. The cold mass, e.g. the main magnet 17 or the cryogen vessel 31, is spaced from the support element 34 via the connecting elements 43 in such a way that a mechanical contact between the displaceable sections 34b and the cold mass 17, 31 is avoided when the displaceable sections 34b are displaced or deflected. In an embodiment, the displaceable sections 34b are deflected or displaced in a radial direction of the support element 34.

The support element 34 is configured in such a way that a predefined relative spatial arrangement of the fixed sections 34a is maintained when the force is applied to the attachment points 44. Thus, a warping or deforming of the cold mass 17, 31 mechanically connected to the support element 34 via the connecting elements 43 can favorably be prevented.

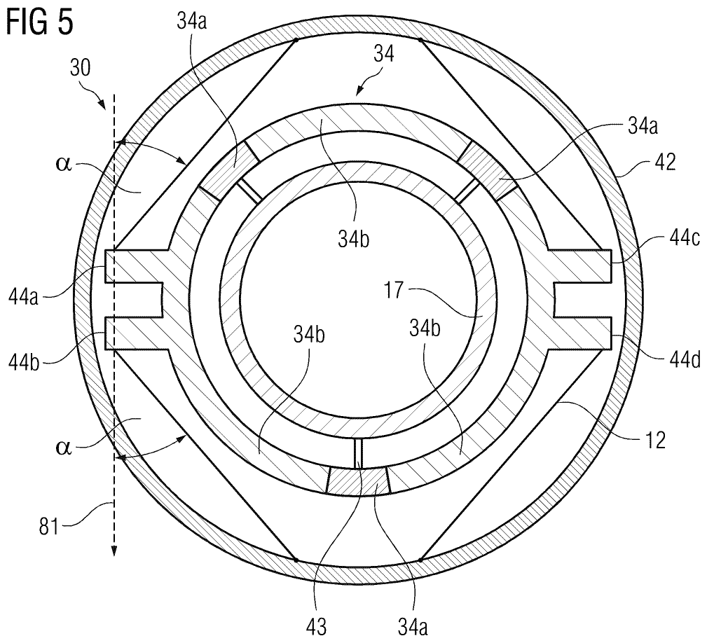

FIG. 5 shows a further embodiment of the magnet support structure 30. In the depicted example, the smallest angle α between the suspension rods 12 and the gravitational force vector 81 is increased in comparison to the embodiment illustrated in FIG. 4. For example, the smallest angle α may be at least 40° or 45°. The shallower angle α of the embodiment shown in FIG. 5 may favorably decrease a tendency of the cold mass 17, 31 to roll and/or twist in relation to the support element 34 in comparison to the embodiment depicted in FIG. 4. A rolling or twisting motion of the cold mass 17, 31 may be caused by a force applied to the attachment points 44 or by electromagnetic forces associated with a magnetic resonance measurement via the magnetic resonance device 11.

Although the fixed sections 34a in FIGS. 4 and 5 are highlighted via a hatched area, the displaceable sections 34b, the fixed sections 34a, and the attachment points 44 may form a monolithic structure. It is also conceivable, however, that the attachment points 44 are mounted to the support structure 34. In an alternate embodiment of the support structure 34, the fixed sections 34a and the displaceable sections 34b are separate components mechanically connected to form the support element 34.

FIG. 6 illustrates an embodiment of the support element 34. In the depicted example, the displaceable sections 34b, the fixed sections 34a and the attachment points 44 form a monolithic structure. The support element 34 comprises the shape of a ring or a tube.

The support element 34 shown in FIG. 6 has been examined in a mechanical strain simulation or load simulation. A force was applied to the attachment points 44a, 44b, 44c and 44d (44a-d) and the resulting displacement of sections of the support structure 34 has been determined and is visualized via the colour axis C. For the simulation, a shock was assumed which corresponded to a force of 5-times the force of gravity. The shock was assumed to accelerate the support element 34 in the Y-direction, to simulate a drop of the support element 34.

FIG. 6 shows an exemplary visualization of a deformation pattern. The attachment points 44b and 44d absorb most of the shock and are displaced by a maximum of about 3 mm. The displaceable sections 34b are affected to a lesser extent, showing a maximum displacement of about 2.2 mm. However, the fixed sections 34a remain in their predefined relative spatial arrangement and show no displacement at all. Thus, a cold mass mechanically connected to the connecting elements 43 arranged at the fixed sections 34a would not be affected by the shock.

The deformation pattern or the information contained in the deformation pattern depicted in FIG. 6 may be used in a computer-implemented method for designing a support element (34) according to an embodiment described above.

The embodiments described herein are to be recognized as examples. It is to be understood that individual embodiments may be extended by or combined with features of other embodiments if not stated otherwise. The embodiments depicted in the FIGS. 1 to 9 are representations that do not necessarily have to be to scale.

Independent of the grammatical term usage, individuals with male, female or other gender identities are included within the term.

What is claimed is:

1. A support assembly for supporting a cold mass of a magnetic resonance device, the support assembly comprising:
an attachment point;
a plurality of displaceable sections; and
a plurality of fixed sections,
wherein each one of the plurality of displaceable sections is configured to be reversibly displaced relative to ones of the plurality of fixed sections when a predefined force is applied to the attachment point,
wherein each one of the plurality fixed sections is configured to remain in a predefined spatial arrangement relative to each other when the predefined force is applied to the attachment point.

2. The support assembly according to claim 1, wherein the support assembly comprises a shape of a ring, a hollow cylinder, a hollow prism, a tube, or a disc.

3. The support assembly according to claim 1, wherein the attachment point is arranged at and is mechanically connected to a displaceable section of the plurality of displaceable sections.

4. The support assembly according to claim 1, further comprising:
a connector configured to mechanically connect to an inert mass,
wherein the connector is arranged at and is mechanically connected to a fixed section of the plurality of fixed sections.

5. The support assembly according to claim 1, wherein the plurality of displaceable sections comprises a first displaceable section and a second displaceable section, and
wherein the first displaceable section and the second displaceable section are separated by a fixed section of the plurality of fixed sections.

6. The support assembly according to claim 1, wherein the plurality of displaceable sections comprises at least three displaceable sections, and
wherein the plurality of fixed sections comprises at least three fixed sections.

7. The support assembly according to claim 1, wherein the support assembly comprises a shape of a tube, a ring, or a disc, and
wherein the attachment point protrudes from a lateral surface of the support assembly.

8. The support assembly according to claim 7, further comprising:
a further attachment point,
wherein the attachment point and the further attachment point are arranged at opposing sides of the support assembly.

9. The support assembly according to claim 1, wherein:
a vector of the predefined force is oriented parallel to a vector of gravitational force,
the support assembly comprises a shape of a tube or a ring, and
each one of the plurality of displaceable sections is configured to be displaced along a radial direction of the support assembly as the predefined force is applied to the attachment point.

10. A magnet support structure, comprising:
an outer vacuum chamber;
a cold mass; and
a support assembly, comprising:
an attachment point;
a plurality of displaceable sections; and
a plurality of fixed sections,
wherein each one of the plurality of displaceable sections is configured to be reversibly displaced relative to ones of the plurality of fixed sections when a predefined force is applied to the attachment point, and
wherein each one of the plurality fixed sections is configured to remain in a predefined spatial arrangement relative to each other when the predefined force is applied to the attachment point,
wherein the attachment point is mechanically connected to the outer vacuum chamber,
wherein the plurality of fixed sections are mechanically connected to the cold mass, and
wherein the cold mass is spaced apart from the plurality of displaceable sections.

11. The magnet support structure according to claim 10, wherein the attachment point is mechanically connected to the outer vacuum chamber via a suspension element.

12. The magnet support structure according to claim 11, wherein a smallest angle between a line defined by an axial extension of the suspension element and a gravitational force vector is at least 35°.

13. The magnet support structure according to claim 10, wherein the plurality of fixed sections are mechanically connected to the cold mass via a plurality of connecting elements, and
wherein the plurality of connecting elements are configured to space the support assembly apart from the cold mass.

14. The magnet support structure according to claim 10, wherein the magnetic support structure is part of a magnetic resonance device configured to acquire magnetic resonance data of an object positioned within an imaging region of the magnetic resonance device.

15. A computer-implemented method for designing a support assembly, comprising:
performing a load simulation of the support assembly to determine a deformation pattern of the support assembly in response to a predefined force being applied to an attachment point,
wherein an inert mass is mechanically connected to the support assembly at a plurality of sections arranged at predefined spatial locations; and
determining a parameter of the support assembly based upon the determined deformation pattern to provide a support assembly comprising a plurality of displaceable sections and a plurality of fixed sections arranged at the predefined spatial locations,
wherein each one of the plurality of displaceable sections is configured to be reversibly displaced relative to ones of the plurality of fixed sections when the predefined force is applied to the attachment point, and
wherein each one of the plurality fixed sections is configured to remain in a predefined spatial arrangement relative to each other when the predefined force is applied to the attachment point.
